(19) United States
(12) Patent Application Publication
(10) Pub. No.: US 2024/0059637 A1
(43) Pub. Date: Feb. 22, 2024

(54) PROCESS AND PLANT FOR PRODUCING METHANOL AND SYNTHESIS GAS

(71) Applicant: L'Air Liquide, Socie'te' Anonyme pour I'Etude et I'Exploitation des Proce'de's Georges Claude, Paris (FR)

(72) Inventors: Tobias OELMANN, Bad Vilbel (DE); Timm Schuhmann, Bensheim (DE); Matthias Linicus, Eppstein (DE)

(21) Appl. No.: 18/234,130

(22) Filed: Aug. 15, 2023

(30) Foreign Application Priority Data
Aug. 17, 2022 (EP) 22190843.7

(51) Int. Cl. C07C 29/152; C01B 3/34; C01B 3/50

(52) U.S. Cl. CPC C07C 29/152; C01B 3/34; C01B 3/503; C01B 2203/061; C01B 2203/0216; C01B 2203/1235; C01B 2203/14; C01B 2203/0405; C01B 2203/1258; C01B 2203/80; C25B 1/04

(57) ABSTRACT

Proposed according to the invention are a process and a plant for producing carbon dioxide-based methanol and synthesis gas, wherein the produced synthesis gas may be utilized process-internally for methanol synthesis. Hydrocarbons which are present in a carbon dioxide input gas stream as an impurity and are inert under the conditions of the methanol synthesis are chemically utilized through integration of a reforming unit, in particular a POX unit. This is done by supplying a purge gas stream diverted from the methanol synthesis loop to said reforming unit in which the hydrocarbons are converted into synthesis gas. In a preferred embodiment the purge gas stream is supplied to a hydrogen recovery unit, in particular a membrane unit. The hydrocarbons-enriched purge gas stream produced on the retentate side is subsequently supplied to the reforming unit.

### CROSS REFERENCE TO RELATED APPLICATIONS

This application claims the benefit of priority under 35 U.S.C. §119 (a) and (b) to European Patent Application No. 22190843.7, filed Aug. 17, 2022, the entire contents of which are incorporated herein by reference.

### BACKGROUND

### Field of the Invention

The invention relates to a process and a plant for producing methanol from carbon dioxide-rich synthesis gas and for producing synthesis gas as a by-product of methanol production, wherein the synthesis gas formed as a by-product may be utilized process-internally for the methanol production.

### Prior Art

On a large industrial scale methanol is produced from synthesis gas. Synthesis gas is typically a mixture of predominantly hydrogen (H₂), carbon monoxide (CO) and carbon dioxide (CO₂). Carbon monoxide and carbon dioxide are often bracketed together under the term “carbon oxides”. Predominantly the following two equilibrium reactions (1) and (2) occur simultaneously over a solid methanol synthesis catalyst.

CO₂+3 H₂ [symbol] CH₃OH+H₂O (1)

CO+2 H₂ [symbol] CH₃OH (2)

Methanol may also be produced from synthesis gas which is low in, or even free from, carbon monoxide. In this case it is predominantly reaction equation (1) that applies in the methanol synthesis. Production of this so-called carbon dioxide-based methanol has a role especially in processes which are to be realized without, or at least with the lowest possible amount of, fossil energy carriers or fossil inputs and which are intended to generate the lowest possible greenhouse gas emissions. An example of such a process is disclosed in WO 2016/034344 A1.

Thus, carbon dioxide-based methanol is producible for example on the basis of carbon dioxide generated by combustion of a fossil input and renewably produced hydrogen. The renewably produced hydrogen is preferably produced by electrolysis of water on the basis of renewably produced electricity.

The carbon dioxide may derive from a very wide variety of sources, for example from offgases from the chemical, steel and cement industries and from biogases and natural gases. The carbon dioxide may be concentrated from the respective gas by carbon capture. Known carbon capture processes used on an industrial scale include physical and chemical absorption in a suitable solvent, cryogenic partial condensation and separation of the gases by membranes. A known physical separation process is the absorption of the carbon dioxide in cryogenic methanol under elevated pressure with subsequent desorption by pressure reduction and optionally stripping with suitable stripping gases which is more or less selective depending on the process mode. A known chemical separation process is the absorption of the carbon dioxide by an amine of an aqueous amine solution with subsequent desorption of the carbon dioxide by heating.

A carbon dioxide stream to be utilized for the methanol synthesis may contain various impurities, in particular hydrocarbons. The hydrocarbons may be composed of various components, for example methane and other lower alkanes (<C₁₀), lower olefins, aromatic compounds, alicyclic compounds and furans.

Before a carbon dioxide stream may be utilized as an input gas stream for a methanol synthesis, purification of the carbon dioxide stream may be necessary. The hydrocarbons may be either separated or decomposed by suitable purification processes. In both cases the hydrogen and carbon bound in the hydrocarbons are not subsequently chemically utilized. If the hydrocarbons are not separated or decomposed these pass into the methanol synthesis as inert components (under the conditions of methanol synthesis) together with the carbon dioxide and hydrogen. Since industrial processes for methanol synthesis always include a synthesis loop with recycling of the process gases, the hydrocarbons accumulate in the circuit and must ultimately be discharged from the circuit with the so-called purge stream. Hydrogen may optionally be separated from the purge stream as value gas and the remaining gas is utilized for example for under-firing in another process/a process upstream of the methanol synthesis. Thus in this case too, at least the carbon bound in the hydrocarbons is not chemically utilized.

### SUMMARY

It is a general object of the present invention to propose a process or a plant which at least partially overcomes the disadvantages of the prior art.

It is an object of the present invention to propose a process or a plant which improves the chemical utilization of the carbon and hydrogen bound in hydrocarbons in respect of the synthesis of methanol.

It is an object of the present invention to propose a process or a plant which improves the chemical utilization of the carbon and hydrogen bound in hydrocarbons in respect of the carbon dioxide-based synthesis of methanol.

The independent claims make a contribution to the at least partial achievement of at least one of the above objects. The dependent claims provide preferred embodiments which contribute to the at least partial achievement of at least one of the objects. Preferred embodiments of constituents of one category according to the invention are, where relevant, likewise preferred for identically named or corresponding constituents of a respective other category according to the invention. The terms “having”, “comprising” or “containing” etc. do not preclude the possible presence of further elements, ingredients etc. The indefinite article “a” does not preclude the possible presence of a plurality.

The abovementioned objects are at least partially achieved by a process for producing methanol and synthesis gas comprising the process steps of:

(a) providing a hydrocarbon-containing carbon dioxide stream;

(b) providing an electrolytically produced hydrogen stream;

(c) combining the streams from steps (a) and (b) to afford a hydrocarbon-containing synthesis gas stream;

(d) reacting the hydrocarbon-containing synthesis gas stream and a recycle gas stream in a methanol synthesis reactor to obtain a raw methanol stream as reaction product and a residual gas stream, wherein the residual gas stream contains synthesis gas unconverted into methanol and hydrocarbons;

(e) separating the residual gas stream into the recycle gas stream and a purge gas stream;

(f) reacting the purge gas stream in the presence of oxygen as oxidant in a reforming step to afford a synthesis gas stream.

According to the invention the carbon and hydrogen bound in the hydrocarbons are chemically utilized by reacting the hydrocarbon-containing purge gas stream in the presence of oxygen to afford a synthesis gas stream in a reforming step. In particular the hydrocarbons of the purge gas stream are reacted in the presence of oxygen to afford a synthesis gas stream in a reforming step. The purge gas stream contains not only the hydrocarbons but also carbon dioxide and hydrogen unconverted in the methanol synthesis reactor. The synthesis gas stream produced by the reforming step contains hydrogen, carbon monoxide and carbon dioxide.

The hydrocarbon-containing synthesis gas stream produced according to step (c) comprises synthesis gas. A synthesis gas is a gas mixture which comprises at least a carbon dioxide (carbon monoxide or carbon dioxide) and hydrogen. The synthesis gas of the hydrocarbon-containing synthesis gas stream produced according to step (c) preferably comprises carbon dioxide and hydrogen. The synthesis gas stream produced according to step (f) comprises synthesis gas. The synthesis gas of the synthesis gas stream produced according to step (f) preferably comprises carbon monoxide, carbon dioxide and hydrogen.

A reforming step is in principle to be understood as meaning the chemical reaction of hydrocarbons with oxygen and/or steam to afford a synthesis gas. According to the reforming step according to step (f) the hydrocarbons of the purge gas stream are reacted at least in the presence of oxygen to afford synthesis gas and optionally reacted in the further presence of steam to afford synthesis gas.

The synthesis gas stream produced by reforming the hydrocarbons of the purge gas stream may be utilized for the methanol synthesis in addition to the hydrocarbon-containing synthesis gas stream.

One embodiment of the process according to the invention is therefore characterized in that the synthesis gas stream produced according to step (f) is reacted in the methanol synthesis reactor in addition to the hydrocarbon-containing synthesis gas stream.

The synthesis gas stream produced according to step (f) comprises carbon monoxide. Since carbon monoxide has a better reactivity towards hydrogen in the methanol synthesis than carbon dioxide the process-internal utilization of the synthesis gas stream produced according to step (f) has a correspondingly advantageous effect.

The reforming step is preferably a partial oxidation. A preferred embodiment of the process according to the invention is therefore characterized in that the reforming step includes a partial oxidation.

The partial oxidation (POX) is preferably performed in a reactor or POX reactor configured therefor. POX reactors may be small and are therefore also suitable for smaller material streams as is regularly the case for the purge gas stream to be reacted.

For methane as the hydrocarbon a partial oxidation initially proceeds according to the following reaction equation (3).

CH₄+1½O₂ [symbol] CO+2 H₂O (3)

The exothermic partial oxidation provides steam and the required heat energy for a subsequent endothermal steam reforming reaction which proceeds uncatalysed according to reaction equation (4).

CH₄+H₂O [symbol] 3 H₂+CO (4)

Steam may additionally be added to the POX reactor to establish an optimal carbon-steam ratio.

The catalysed variant of equation (4) is referred to as autothermal reforming and this is to be distinguished from the “pure” partial oxidation in a POX reactor.

The steam reforming reaction is accompanied by the water gas shift reaction according to reaction equation (5) which is exothermic.

CO+H₂O [symbol] CO₂+H₂ (5)

The main elements of a POX reactor are a burner and a combustion chamber which are arranged in a refractory-lined pressure vessel. In a POX reactor the partial oxidation of the hydrocarbon-containing input stream always occurs as a result of substoichiometric amounts of oxygen. The temperature of the gas mixture at the outlet of the reactor is typically in the range from 1250° C. to 1450° C. The process pressure is typically 20 to 100 bar.

According to the invention a purification of the carbon dioxide stream in respect of the hydrocarbons present in the carbon dioxide stream is not necessary since these are chemically utilized in the context of step (f). This is why according to the invention step (a) comprises providing a hydrocarbon-containing carbon dioxide stream. In addition, step (b) comprises providing an electrolytically produced hydrogen stream. Both streams are combined, thus resulting in a hydrocarbon-containing synthesis gas stream according to (c). This synthesis gas stream contains at least carbon dioxide and hydrogen and hydrocarbons as an impurity. This synthesis gas stream can also contain carbon monoxide.

In one example the proportion of hydrocarbons in the hydrocarbon-containing carbon dioxide stream is 0.1% to 10% by volume or 1% to 10% by volume or 2% to 8% by volume. The proportion of carbon dioxide in the hydrocarbon-containing carbon dioxide stream may vary markedly depending on whether the carbon dioxide stream has been concentrated in a preceding process step, in particular in a carbon capture unit. Accordingly, the proportion of carbon dioxide in the hydrocarbon-containing carbon dioxide stream may be for example in a range from 50% by volume to 99.5% by volume. The hydrocarbon-containing carbon dioxide stream thus preferably has a carbon dioxide proportion of 50% by volume to 99.5% by volume or a carbon dioxide proportion of 75% by volume to 99.5% by volume or a carbon dioxide proportion of 90% by volume to 99.5% by volume or a carbon dioxide proportion of 95% by volume to 99.5% by volume.

The hydrocarbon-containing synthesis gas stream produced according to step (c) is to be distinguished from the synthesis gas stream produced by step (f). To this end the hydrocarbon-containing synthesis gas stream produced according to step (c) may be referred to as “first synthesis gas stream” and the synthesis gas stream produced according to step (f) may be referred to as “second synthesis gas stream”.

Step (d) comprises reacting the hydrocarbon-containing synthesis gas stream and a recycle gas stream in a methanol synthesis reactor. It especially comprises reacting the components hydrogen and carbon dioxide and optionally carbon monoxide that are present in the hydrocarbon-containing synthesis gas stream and the recycle gas stream to afford raw methanol as a desired reaction product.

The reaction in the methanol synthesis reactor is carried out at synthesis pressure, for example a pressure of 40 bar to 90 bar, preferably 60 bar to 85 bar. Typical pressure ranges used in the methanol synthesis are well known to those skilled in the art. To achieve the required synthesis pressure the hydrocarbon-containing synthesis gas stream and the recycle gas stream are compressed to the required pressure by one or more compressor stages.

Raw methanol contains methanol and water and optionally undesired by-products. The raw methanol stream is separated from a residual gas stream (which is uncondensable under the prevailing conditions) in the context of a gas-liquid separation especially by cooling, condensation and separation. The raw methanol stream is accordingly discharged from the methanol synthesis reactor in particular and subjected to a further processing operation.

The methanol synthesis reactor includes a suitable methanol synthesis catalyst, in particular a solid copper-based catalyst. Suitable catalysts are well known to those skilled in the art.

The methanol synthesis reactor may comprise one or more reactor stages. If two or more reactor stages are arranged in series each reactor stage may be followed by an intermediate condensation of the raw methanol stream or in this case of a raw methanol substream.

The reaction in the methanol synthesis reactor affords a raw methanol stream as reaction product and a residual gas stream. The residual gas stream contains synthesis gas unconverted into methanol and also hydrocarbons since the latter do not react under the conditions of methanol synthesis, i.e. are inert under these conditions.

The residual gas stream is in particular divided into a recycle gas stream and a purge gas stream downstream of the methanol synthesis reactor. The recycle gas stream is in particular recycled to the inlet of the methanol synthesis reactor and preferably combined with the hydrocarbon-containing synthesis gas stream before both streams are reacted in the methanol synthesis reactor together. The purge gas stream is not part of this circuit but rather is in particular discharged from the methanol synthesis loop downstream of the methanol synthesis reactor and reacted according to step (f).

In the course of reacting the purge gas stream in the presence of oxygen as oxidant in a reforming step to afford a synthesis gas stream it may be provided that the purge gas stream is supplied with a steam stream so that the purge gas stream and the steam stream are reacted to afford the synthesis gas stream in the reforming step according to step (f). The supply of steam makes it possible to establish an optimal carbon-steam ratio, for example for a partial oxidation.

The electrolytically produced hydrogen stream is especially provided by an electrolyzer. Depending on the process mode a portion of the electrolytically produced hydrogen stream is supplied to the hydrocarbon-containing carbon dioxide stream or the entire electrolytically produced hydrogen stream is supplied to the hydrocarbon-containing synthesis gas stream. The electrolyzer is preferably configured such that the entire electrolytically produced hydrogen stream is supplied to the hydrocarbon-containing carbon dioxide stream. The electrolyzer may then be operated under full load for most of the time. The electrolyzer is then preferably configured such that the resulting hydrocarbon-containing synthesis gas stream has a stoichiometry number suitable for the methanol synthesis, for example a stoichiometry number SN of 1.9 to 2.5, preferably of 2.0 to 2.4, wherein

$$S N = \frac{n ( {H}_{2} ) - n ( {CO}_{2} )}{n ( CO ) + n ( {CO}_{2} )} , with n in [ mol ] .$$

The electrolytically produced hydrogen stream may be produced by any electrolysis process familiar to those skilled in the art. The electrolysis process is preferably a water electrolysis. Examples include alkaline electrolysis, proton exchange membrane electrolysis (PEM electrolysis), anion exchange membrane electrolysis (AEM electrolysis), high-temperature electrolysis (HTE) and electrolysis using solid oxide electrolyzer cells (SOEC).

One embodiment of the process according to the invention is characterized in that before the reacting according to step (f) the purge gas stream is supplied to a hydrogen recovery unit to obtain a hydrocarbons-enriched purge gas stream and a hydrogen-rich stream.

Thus in this embodiment the hydrocarbons-enriched purge gas stream is reacted to afford a synthesis gas stream according to step (f).

The separation of the hydrogen has the result that no hydrogen or only minimal hydrogen amounts from the purge gas stream are subjected to the reforming step according to step (f). Hydrogen passing into the reforming step may be converted into water due to the presence of oxygen, thus potentially leading to chemical losses of hydrogen. If the hydrogen is separated beforehand and subsequently supplied to the hydrocarbon-containing synthesis gas stream as produced according to step (c), the losses of hydrogen in this respect are minimized.

In one embodiment the hydrogen-rich stream is therefore supplied to the hydrocarbon-containing synthesis gas stream.

One embodiment of the process according to the invention is characterized in that the hydrogen recovery unit comprises a membrane unit, wherein the hydrocarbons-enriched purge gas stream is produced on the retentate side of the membrane unit and the hydrogen-rich stream is produced on the permeate side of the membrane unit.

The use of a membrane unit has the advantage that the hydrocarbons-enriched purge gas stream is produced on the retentate side of the membrane unit without significant pressure losses. This stream may therefore be supplied to the reforming step according to step (f) without additional compression. If the methanol synthesis is operated at 75 bar for example and assuming a pressure drop of 5 bar over the retentate side of the membrane unit including pipe conduits, the hydrocarbons-enriched purge gas stream has a pressure of about 70 bar. Such a pressure is suitable for direct introduction into a POX reactor for example. This advantage is not realizable when using a pressure swing adsorption apparatus (PSA) since the desorbing of the “heavy” components from the PSA fixed bed, here the hydrocarbons, always requires the pressure to be reduced.

One embodiment of the process according to the invention is characterized in that the hydrocarbon-containing carbon dioxide stream is treated in a hydrodesulfurization unit for removal of sulfur compounds before the combining according to step (c).

Since sulfur compounds are catalyst poisons for methanol synthesis catalysts it may be necessary to remove these sulfur compounds in a hydrodesulfurization unit depending on the impurity profile of the hydrocarbon-containing carbon dioxide stream.

It is preferable to this end to employ the hydrogen-rich stream for the hydrogenation in the hydrodesulfurization unit.

The hydrogen-rich stream separated from the purge gas stream by a hydrogen recovery unit may also be supplied to the methanol synthesis reactor though this requires compression of this stream to synthesis pressure. This hydrogen-rich stream is ultimately obtained at low pressure irrespective of whether production thereof employed a membrane unit or a PSA.

Since the hydrogenation of sulfur compounds in a hydrodesulfurization unit is typically or preferably performed at low pressure an additional compression of the hydrogen-rich stream is in this case and advantageously required at least only to a small extent, if at all.

If the hydrogen-rich stream is supplied to the methanol synthesis reactor the hydrogen-rich stream is preferably supplied to the electrolytically produced hydrogen stream before the combining according to step (c) and/or to the hydrocarbon-containing synthesis gas stream before the reacting according to step (d).

The hydrogen-enriched hydrocarbon-containing synthesis gas stream obtained in both cases is preferably subsequently compressed to synthesis pressure.

One embodiment of the process according to the invention is characterized in that the methanol synthesis reactor includes a water-cooled reactor stage, wherein the cooling by the water-cooled reactor stage produces steam and the steam is utilized as process steam for the reforming step according to step (f).

Methanol synthesis reactors may include water-cooled or gas-cooled reactor stages. Water-cooled reactor stages often employ boiling boiler feed water as cooling medium or in other words medium for temperature control of the exothermic methanol formation reaction. Steam is accordingly formed at the outlet of the cooling system of such a reactor stage. This steam may advantageously be utilized as process steam for the reforming step according to step (f). In such a case the hydrocarbon-containing purge gas stream is reacted in the presence of oxygen and steam to afford the synthesis gas stream.

One embodiment of the process according to the invention is characterized in that an electrolytically produced oxygen stream is provided and the oxygen of the electrolytically produced oxygen stream is utilized as oxidant in the reforming step according to step (f).

The use of electrolytically produced oxygen as oxidant has the advantage that oxygen produced in this way is of high purity. A corresponding purity is otherwise achieved only by obtaining oxygen by cryogenic air separation. However, such an air separator is demanding in terms of apparatus and energy requirements and may not necessarily be available. However, a water electrolyzer always forms oxygen as a “by-product” which is normally not utilized. It is therefore preferable when the electrolytically produced hydrogen stream and the electrolytically produced oxygen stream are provided by the same electrolyzer. If air or oxygen-enriched air is used as oxidant this further has the disadvantage that, due the nitrogen present, undesired nitrogen oxides can always also be formed in the reforming step.

A further advantage of the use of an electrolytically produced oxygen stream is that residual hydrogen amounts present in this oxygen stream need not be removed. The presence of hydrogen in an electrolytically produced oxygen stream is unavoidable due to diffusion processes via the membranes or diaphragms between the anodic and cathodic regions of the electrolysis cells. Since hydrogen and water also unseparated from the oxygen stream are not disruptive in the reforming step according to step (f) a corresponding purification of the electrolytically produced oxygen stream may be dispensed with.

One embodiment of the process according to the invention is characterized in that the hydrocarbon-containing carbon dioxide stream is provided by a carbon capture unit.

This generally markedly increases the purity in respect of carbon dioxide of a primary carbon dioxide stream. Examples of primary carbon dioxide streams are offgas streams or flue gas streams. A corresponding hydrocarbon-containing carbon dioxide stream is thus more suitable for the subsequent methanol synthesis since the formation of by-products is avoided. The carbon capture unit may be any apparatus or plant known to those skilled in the art which makes it possible to form a purified carbon dioxide stream. This especially includes a gas scrubbing process in which carbon dioxide is initially absorbed in a solvent more or less selectively, either physically or chemically, and is subsequently desorbed again through alteration of the physical conditions such as pressure and/or temperature or the introduction of desorption media.

One embodiment of the process according to the invention is characterized in that the raw methanol stream produced according to step (d) is separated into pure methanol and water by a thermal separation process, and wherein the thermal separation process separates hydrocarbons as a by-product stream and the resulting by-product stream is supplied to the purge gas stream to react the purge gas stream and the by-product stream in the reforming step according to step (f) to afford the synthesis gas stream.

The raw methanol formed and discharged from the methanol synthesis reactor is typically separated into water and methanol in a thermal separation apparatus, for example a rectification column. Since hydrocarbons present in the hydrocarbon-containing synthesis gas stream are inert under the conditions of the methanol synthesis, hydrocarbons condensed with the raw methanol are especially entrained into the thermal separation apparatus and require separation therein. The hydrocarbons separated as a by-product stream here can also advantageously be supplied to the purge gas stream for the subsequent reaction to afford synthesis gas according to step (f). The chemical utilization of the hydrocarbons is thus further improved.

One embodiment of the process according to the invention is characterized in that a purge gas substream is separated from the purge gas stream and the purge gas substream is discharged from the process.

This may be necessary when certain inert components accumulate in the process, in particular in the methanol synthesis loop, also as a result of the reaction of the purge gas stream in the reforming step according to step (f) and the synthesis gas stream produced by step (f) is supplied to the methanol synthesis. Said compounds may be inert compounds such as molecular nitrogen for example.

The abovementioned objects are further at least partially achieved by a plant for producing methanol and synthesis gas comprising the following plant components in operative connection with one another:
(a) means configured for providing a hydrocarbon-containing carbon dioxide stream;
(b) an electrolyzer configured for providing an electrolytically produced hydrogen stream;
(c) means configured for combining the hydrocarbon-containing carbon dioxide stream and the electrolytically produced hydrogen stream, by means of which a hydrocarbon-containing synthesis gas stream is obtainable;
(d) a methanol synthesis reactor configured for reacting the hydrocarbon-containing synthesis gas stream and a recycle gas stream, by means of which a raw methanol stream as reaction product and a residual gas stream are obtainable, wherein the residual gas stream contains synthesis gas unconverted into methanol and hydrocarbons;
(e) means configured for separating the residual gas stream into the recycle gas stream and a purge gas stream;
(f) a reactor configured for reacting the purge gas stream in the presence of oxygen as oxidant in a reforming step to afford a synthesis gas stream.

One embodiment of the plant according to the invention comprises a hydrogen recovery unit arranged upstream of the reactor (f) and means configured for supplying the purge gas stream to said hydrogen recovery unit, wherein the hydrogen recovery unit is configured for producing a hydrocarbons-enriched purge gas stream and a hydrogen-rich stream and wherein the plant comprises means for supplying the hydrocarbons-enriched purge gas stream to the reactor (f).

One embodiment of the plant according to the invention is characterized in that the hydrogen recovery unit comprises a membrane unit, wherein the membrane unit is configured such that the hydrocarbons-enriched purge gas stream is producible on the retentate side of the membrane unit and the hydrogen-rich stream is producible on the permeate side of the membrane unit.

One embodiment of the plant according to the invention further comprises a hydrodesulfurization unit, wherein the hydrodesulfurization unit is configured for removing sulfur compounds from the hydrocarbon-containing carbon dioxide stream.

One embodiment of the plant according to the invention is characterized in that the plant comprises means configured for utilizing the hydrogen-rich stream for the hydrogenation in the hydrodesulfurization unit.

One embodiment of the plant according to the invention is characterized in that the plant comprises means for supplying the hydrogen-rich stream to the electrolytically produced hydrogen stream and/or means configured for supplying the hydrogen-rich stream to the hydrocarbon-containing synthesis gas stream.

One embodiment of the plant according to the invention is characterized in that the reactor (f) is configured as a POX reactor.

One embodiment of the plant according to the invention is characterized in that the methanol synthesis reactor includes a water-cooled reactor stage, wherein steam is producible in the cooling by the water-cooled reactor stage and the plant comprises means configured for utilizing the steam as process steam in reactor (f).

One embodiment of the plant according to the invention is characterized in that the electrolyzer is configured for providing an electrolytically produced oxygen stream and the plant comprises means for utilizing the electrolytically produced oxygen stream as oxidant in reactor (f).

One embodiment of the plant according to the invention is characterized in that the plant comprises a carbon capture unit configured for providing the hydrocarbon-containing carbon dioxide stream.

One embodiment of the plant according to the invention is characterized in that the plant comprises a thermal separation apparatus configured for separating the raw methanol stream into pure methanol and water, and wherein a hydrocarbon-containing by-product stream is producible by the thermal separation apparatus and the plant comprises means configured for supplying the by-product stream to the purge gas stream, as a result of which the purge gas stream and the by-product stream may be reacted to afford the synthesis gas stream in reactor (f). One embodiment of the plant according to the invention is characterized in that the plant comprises means configured for reacting the synthesis gas stream producible according to (f) as well as for reacting the hydrocarbon-containing synthesis gas stream in the methanol synthesis reactor (d). One embodiment of the plant according to the invention is characterized in that the plant comprises means for separating a purge gas substream from the purge gas stream and the plant comprises means for discharging the purge gas substream from the process.

### BRIEF DESCRIPTION OF THE DRAWINGS

The invention is more particularly elucidated hereinbelow by way of working examples and numerical examples without in any way limiting the subject-matter of the invention. Further features, advantages and possible applications of the invention will be apparent from the following description of the working examples in connection with the figures and the numerical examples. In the figures, functionally and/or structurally identical or at least similar elements are given identical reference numerals.

In the figures:

FIG. 1 shows a block flow diagram of an inventive process for producing methanol and synthesis gas according to a first working example,

FIG. 2 shows a block flow diagram of an inventive process for producing methanol and synthesis gas according to a second working example.

### DESCRIPTION OF THE PREFERRED EMBODIMENTS

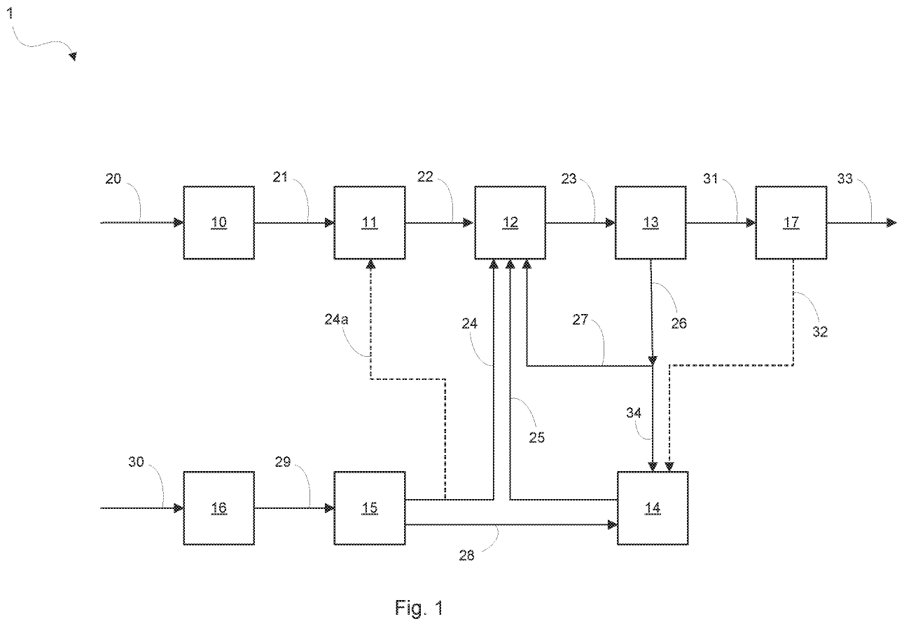

FIG. 1 shows a highly simplified block flow diagram of a process 1 for producing methanol and synthesis gas according to a first working example of the invention.

A hydrocarbon-containing and carbon dioxide-containing input gas stream 20, for example an offgas or flue gas, is treated in a carbon capture unit 10 to increase the carbon dioxide concentration in the input gas stream 20. The resulting hydrocarbon-containing carbon dioxide stream 21 still contains sulfur compounds and is therefore treated in a hydrodesulfurization unit 11 to achieve quantitative removal of the sulfur compounds from the stream 21. This results in a sulfur-free hydrocarbon-containing carbon dioxide stream 22 which is combined with an electrolytically produced hydrogen stream 24, a synthesis gas stream 25 and a recycle gas stream 27 and compressed in a compression unit 12 to a synthesis pressure suitable for the production of methanol. The resulting stream 23 is a combined stream of a hydrocarbon-containing synthesis gas stream (generated from streams 22, 24 and 25) and the recycle gas stream 27.

Conduit 30 is used to provide a raw water stream 30 which is treated, for example demineralized, in a water treatment apparatus 16 to afford a pure water stream 29 which is supplied to an electrolyzer 15. Electrolyzer 15 is for example a PEM electrolyzer which generates from pure water as the electrolysis medium an anodically produced oxygen stream 28 and a cathodically produced hydrogen stream 24. The hydrogen stream 24 is combined with the hydrocarbon-containing (sulfur-free) carbon dioxide stream 22 to afford a hydrocarbon-containing synthesis gas stream. This hydrocarbon-containing synthesis gas stream is supplemented by the synthesis gas stream 25 produced in a POX reactor 14. The electrolytically produced oxygen stream 28 is utilized as oxidant in the POX reactor 14. The electrolyzer 15 may optionally be a high-pressure electrolyzer which is operated at 25 bar for example. If the POX reactor 14 is operated at low enough pressure, for example 20 bar, it is advantageously possible to dispense with compression of the oxygen stream 28. A portion of the electrolytically produced hydrogen stream 24 may be diverted as substream 24a and supplied to the hydrodesulfurization unit 11. The electrolytically produced hydrogen substream 24a is utilized therein for the hydrogenation.

The combined stream 23 contains hydrogen, carbon dioxide, carbon monoxide and hydrocarbons and is supplied to a methanol synthesis reactor 13 in which the abovementioned constituents, with the exception of the hydrocarbons, are reacted over a suitable methanol synthesis catalyst to afford raw methanol. Raw methanol contains at least methanol and water. Since the reaction in the methanol synthesis reactor 13 is incomplete due to the establishment of a thermodynamic equilibrium, not only a raw methanol stream 31 but also a residual gas stream 26 are discharged from the methanol synthesis reactor. The residual gas stream 26 contains unconverted synthesis gas constituents and the hydrocarbons inert under the conditions of methanol synthesis. The residual gas stream 26 is divided into a purge gas stream 34 and a recycle gas stream 27. The recycle gas stream 27 is part of the methanol synthesis loop which is formed at least by the streams 23, 26 and 27 and the methanol synthesis reactor 13 and the compression unit 12.

The purge gas stream 34 is discharged from this loop, i.e. this recirculating process. Said stream is subsequently supplied to the POX reactor 14 in which a reforming of the hydrocarbons of the purge gas stream is carried out using the electrolytically produced oxygen stream 28 and with a steam stream (not shown). Reforming the hydrocarbons produces a synthesis gas stream 25 which contains hydrogen, carbon monoxide and carbon dioxide. This synthesis gas stream 25 is supplemented by the hydrocarbon-containing synthesis gas stream obtained by combination of the streams 22 and 24. The hydrocarbons present in the stream 22 are thus chemically utilized for the methanol production in the methanol synthesis reactor 13. Before the synthesis gas stream 25 is supplied to the compression unit 12 it passes through a cooling sector which comprises for example a waste heat boiler (not shown). Since the waste heat boiler generally produces high pressure steam at about 40 bar this steam may be used for improving process integration, for example as turbine propulsion steam in the compression unit 12 or as heating medium in the thermal separation apparatus 17 arranged downstream of the methanol synthesis reactor.

The raw methanol stream 31 is supplied to a thermal separation apparatus 17, here a rectification column 17. The rectification 17 produces a pure methanol stream 33, for example a pure methanol stream having a content of at least 99% by weight of methanol or of at least 99.5% by weight of methanol. A further “product” of the thermal separation generated in the rectification column 17 is water which may be utilized for improving process integration, for example as raw water stream 30 (not shown). The rectification column 17 also generates a hydrocarbon-containing by-product stream 32 which may optionally likewise be supplied to the POX reactor 14 for reaction to afford synthesis gas to further increase the carbon yield of the process.

Since the synthesis gas stream 25 is entirely supplied to the methanol synthesis loop as a reactant stream it may be necessary to divert a purge gas substream from the purge gas stream 34 and discharge said substream from the process (not shown). This prevents components also unconverted in the POX reactor, i.e. inert components, from passing into the methanol synthesis loop and accumulating there.

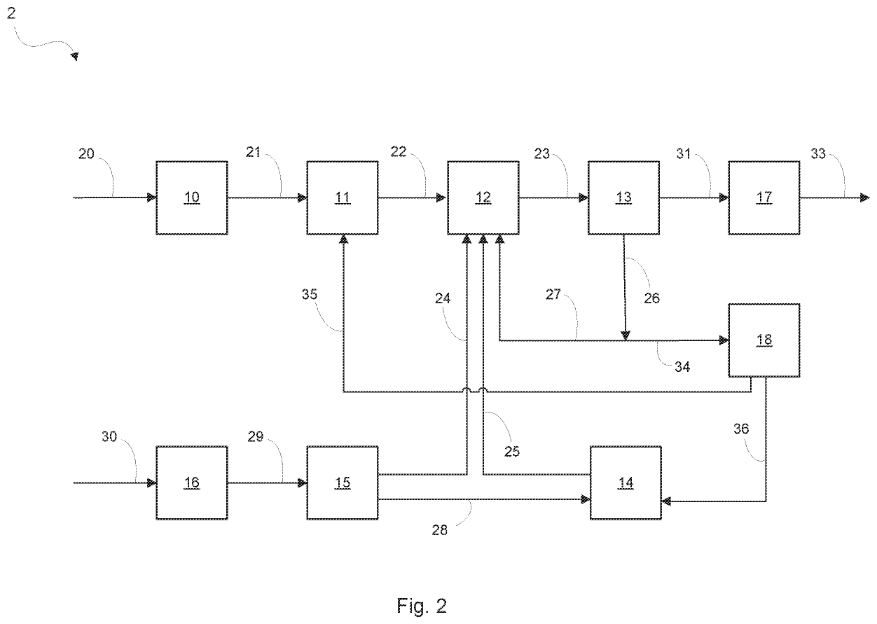

FIG. 2 shows a highly simplified block flow diagram of a process 2 for producing methanol and synthesis gas according to a second working example of the invention.

Process 2 according to FIG. 2 differs from process 1 according to FIG. 1 in particular via the presence of an additional hydrogen recovery unit 18. The hydrogen recovery unit 18 is in particular a membrane unit 18. The hydrocarbon-containing purge gas stream 34 is treated in this membrane unit 18 before it is supplied to the POX reactor 14. The membrane unit 18 produces a hydrogen stream 35 on the permeate side and a correspondingly hydrocarbons-enriched (or vice versa hydrogen-depleted) purge gas stream 36 on the retentate side. This hydrocarbons-enriched purge gas stream 36 is supplied to the POX reactor 14. Since stream 36 is free from hydrogen or at least markedly depleted in hydrogen, hydrogen accordingly only undergoes reaction with oxygen in the POX reactor to afford water to a small extent, if at all, thus further improving the hydrogen yield of the overall process. Instead, the hydrogen stream 35 separated on the permeate side is supplied to the hydrodesulfurization unit 11. This especially has the advantage that the hydrogen stream 35 need not be compressed to be employed as hydrogenating agent in the hydrodesulfurization unit 11. As an alternative the hydrogen stream 35 could also be compressed in the compression unit 12 and would then form a portion of the synthesis gas converted into methanol.

Since the hydrocarbons-enriched purge gas stream 36 is produced on the retentate side in the membrane unit 18, said stream does not exhibit a significant pressure drop relative to the pressure in the methanol synthesis reactor 13. Stream 36 therefore need not be specifically compressed before being introduceable into the POX reactor 14.

The following numerical examples are based on simulation data and serve to further elucidate the invention. The simulation data were generated using the software AspenPlus®.

The following table initially shows an example of a typical concentration of hydrocarbons in the hydrocarbon-containing carbon dioxide stream 22 and how this changes in the process. The example assumes a carbon dioxide stream comprising a proportion of 5% by volume of methane.

| | Stream 22 | Stream 22 + 24 | Stream 27 | Stream 34 |
|---|---|---|---|---|
| Proportion of | 5.00 | 1.30 | 17.2 | 17.2 |
| methane/vol % | | | | |
| Normalized | 100 | 386 | 1737 | 26 |
| volume flow/% | | | | |
| Pressure/bara | 1.0 | 80.0 | 75.0 | 75.0 |

The original concentration of 5% by volume of methane in the hydrocarbon-containing carbon dioxide stream 22 is reduced to 1.30% by volume by addition of the electrolytically produced hydrogen stream 25. However a marked enrichment in methane occurs due to the reaction to afford methanol in the methanol synthesis loop as is discernible from the concentrations of 17.2% by volume for the recycle gas stream 27 and the purge gas stream 34. This proportion is significantly higher than in stream 22 or in combined stream 22+24. Due to the low volume flow of the purge gas stream 34 said stream is suitable as an input gas stream for the POX reactor since said reactor can therefore be made small and thus particularly efficient and also cost-effective.

For the following comparative examples it is assumed that hydrocarbons from the purge gas stream 34 and hydrocarbons from the byproduct stream 32 are entirely burnt instead of being utilized according to the invention. The normalized carbon dioxide emissions for the comparative examples are reported on this basis and set at 100%.

The following table shows the comparison of a comparative Example 1 with an inventive Example 1 for the integration of a POX reactor for the purge gas stream 34 at 5% by volume of methane in stream 22.

| | Comparative | |
|---|---|---|
| | Example 1 | Example 1 |
| Normalized methanol capacity of the plant | 100% | 105% |
| Normalized carbon dioxide emission | 100% | 23% |

The utilization of the hydrocarbons of the purge gas stream to produce synthesis gas via the POX reactor and subsequent utilization of this synthesis gas in the methanol synthesis accordingly saves 77% of the carbon dioxide emissions. The methanol capacity of the plant is further increased by 5% through the chemical utilization of the bound carbon and hydrogen.

The following table shows the same case for a proportion of 10% by volume of methane in stream 22.

| | Comparative | |
|---|---|---|
| | Example 2 | Example 2 |
| Normalized methanol capacity of the plant | 100% | 117% |
| Normalized carbon dioxide emission | 100% | 22% |

The following table shows the same case for a proportion of 2.5% by volume of methane and 2.5% by volume of further hydrocarbons in stream 22. The further hydrocarbons have the following composition: 1% by volume ethane, 0.75% by volume propane, 0.5% by volume butane and 0.25% by volume pentane.

| | Comparative | |
|---|---|---|
| | Example 3 | Example 3 |
| Normalized methanol capacity of the plant | 100% | 105% |
| Normalized carbon dioxide emission | 100% | 25% |

The following table shows the advantage over Example 3 if the purge gas stream 34 is additionally treated in a membrane unit 18 and the hydrocarbons-enriched purge gas stream 36 obtained at the retentate side is supplied to the POX reactor 14.

| | Comparative | |
|---|---|---|
| | Example 4 | Example 4 |
| Normalized methanol capacity of the plant | 100% | 109% |
| Normalized carbon dioxide emission | 100% | 21% |

Apart from the abovementioned advantages it has surprisingly been found with regard to Example 4 that the additional membrane unit 18 makes it possible to further increase the methanol capacity relative to Example 3 and reduce the emissions.

### LIST OF REFERENCE SYMBOLS

1, 2 Process
10 Carbon capture unit
11 Hydrodesulfurization unit
12 Compression unit
13 Methanol synthesis reactor
14 POX reactor
15 Electrolyzer
16 Water treatment apparatus
17 Rectification
18 Hydrogen recovery unit (membrane unit)
20 Input gas stream
21 Hydrocarbon-containing carbon dioxide stream (sulfur-containing)
22 Hydrocarbon-containing carbon dioxide stream (sulfur-free)
23 Combined stream of hydrocarbon-containing synthesis gas stream and recycle gas stream
24, 24a Electrolytically produced hydrogen stream
25 Synthesis gas stream
26 Residual gas stream
27 Recycle gas stream
28 Electrolytically produced oxygen stream
29 Ultrapure water stream
30 Raw water stream
31 Raw methanol stream
32 Byproduct stream
33 Pure methanol stream
34 Purge gas stream
35 Hydrogen stream
36 Hydrocarbons-enriched purge gas stream

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A process for producing methanol and synthesis gas comprising:
(a) providing a hydrocarbon-containing carbon dioxide stream;
(b) providing an electrolytically produced hydrogen stream;
(c) combining the streams from steps (a) and (b) thereby producing a hydrocarbon-containing synthesis gas stream;
(d) reacting the hydrocarbon-containing synthesis gas stream and a recycle gas stream in a methanol synthesis reactor thereby producing a raw methanol stream as reaction product and a residual gas stream, wherein the residual gas stream contains synthesis gas unconverted into methanol and hydrocarbons;
(e) separating the residual gas stream into the-recycle gas stream and a purge gas stream;
(f) reacting the purge gas stream in the presence of oxygen as oxidant in a reforming step to afford a synthesis gas stream.

2. The process according to claim 1, wherein before the reacting according to step (f) the purge gas stream is supplied to a hydrogen recovery unit thereby producing a hydrocarbons-enriched purge gas stream and a hydrogen-rich stream.

3. The process according to claim 2, wherein the hydrogen recovery unit comprises a membrane unit, wherein the hydrocarbons-enriched purge gas stream is produced on the retentate side of the membrane unit and the hydrogen-rich stream is produced on the permeate side of the membrane unit.

4. The process according to claim 1, wherein the hydrocarbon-containing carbon dioxide stream is treated in a hydrodesulfurization unit for removal of sulfur compounds before the combining according to step (c).

5. The process according to claim 2, wherein the hydrogen-rich stream is utilized for the hydrogenation in the hydrodesulfurization unit.

6. The process according to claim 2, wherein the hydrogen-rich stream is supplied to the electrolytically produced hydrogen stream before the combining according to step (c) and/or to the hydrocarbon-containing synthesis gas stream before the reacting according to step (d).

7. The process according to claim 1, wherein the reforming step includes a partial oxidation.

8. The process according to claim 1, wherein the methanol synthesis reactor includes a water-cooled reactor stage, wherein the cooling by the water-cooled reactor stage produces steam and the steam is utilized as process steam for the reforming step according to step (f).

9. The process according to claim 1, wherein an electrolytically produced oxygen stream is provided and the oxygen of the electrolytically produced oxygen stream is utilized as oxidant in the reforming step according to step (f).

10. The process according to claim 1, wherein the hydrocarbon-containing carbon dioxide stream is provided by a carbon capture unit.

11. The process according to claim 1, wherein the raw methanol stream produced according to step (d) is separated into pure methanol and water by a thermal separation process, and wherein the thermal separation process separates hydrocarbons as a byproduct stream and the resulting byproduct stream is supplied to the purge gas stream to react the purge gas stream and the byproduct stream in the reforming step according to step (f) to afford the synthesis gas stream.

12. The process according to claim 1, wherein the synthesis gas stream produced according to step (f) is reacted in the methanol synthesis reactor in addition to the hydrocarbon-containing synthesis gas stream.

13. A plant for producing methanol and synthesis gas comprising the following plant components in operative connection with one another:
(a) a means configured for providing a hydrocarbon-containing carbon dioxide stream;
(b) an electrolyzer configured for providing an electrolytically produced hydrogen stream;
(c) a means configured for combining the hydrocarbon-containing carbon dioxide stream and the electrolytically produced hydrogen stream, by means of which a hydrocarbon-containing synthesis gas stream is obtainable;
(d) a methanol synthesis reactor configured for reacting the hydrocarbon-containing synthesis gas stream and a recycle gas stream, by means of which a raw methanol stream as reaction product and a residual gas stream are obtainable, wherein the residual gas stream contains synthesis gas unconverted into methanol and hydrocarbons;
(e) a means configured for separating the residual gas stream into the recycle gas stream and a purge gas stream;
(f) a reactor configured for reacting the purge gas stream in the presence of oxygen as oxidant in a reforming step to afford a synthesis gas stream.

14. The plant according to claim 13, further comprising a hydrogen recovery unit arranged upstream of the reactor (f) and means configured for supplying the purge gas stream to said hydrogen recovery unit, wherein the hydrogen recovery unit is configured for producing a hydrocarbons-enriched purge gas stream and a hydrogen-rich stream and wherein the plant comprises means for supplying the hydrocarbons-enriched purge gas stream to the reactor (f).

15. The plant according to claim 14, wherein the hydrogen recovery unit comprises a membrane unit, wherein the membrane unit is configured such that the hydrocarbons-enriched purge gas stream is producible on the retentate side of the membrane unit and the hydrogen-rich stream is producible on the permeate side of the membrane unit.

16. The plant according to claim 13, wherein the reactor (f) is configured as a POX reactor.

17. The plant according to claim 13, wherein the plant comprises means configured for reacting the synthesis gas stream producible according to (f) in addition to reacting the hydrocarbon-containing synthesis gas stream in the methanol synthesis reactor (d).
